(19) United States
(12) Patent Application Publication
(10) Pub. No.: US 2012/0121666 A1
(43) Pub. Date: May 17, 2012

(54) COMPOSITIONS OF PARTICLES

(75) Inventors: Jimmie R. Baran, JR., Prescott, WI (US); Madeline P. Shinbach, St. Paul, MN (US)

(73) Assignee: 3M Innovative Properties Company

(21) Appl. No.: 13/357,083

(22) Filed: Jan. 24, 2012

Related U.S. Application Data
(63) Continuation of application No. 12/605,849, filed on Oct. 26, 2009.
Application No. 12/605,849 is a continuation of application No. 11/615,516, filed on Dec. 22, 2006, now abandoned.

(51) Int. Cl. A61K 9/14; A61K 47/02; A61K 47/06; C08K 3/40; C08K 3/22; C08K 5/00; C04B 35/00; A23L 1/00; A61K 47/04; C08K 3/36; B82Y 5/00

(52) U.S. Cl. 424/400; 514/770; 514/769; 514/772; 106/400; 106/489; 106/482; 106/456; 106/425; 106/450; 106/499; 501/1; 501/153; 501/154; 501/134; 426/531; 977/773; 977/906

(57) ABSTRACT

Method of making particle compositions exhibiting improved floodability and/or flowability properties. The compositions generally contain particles and non-surface modified nanoparticles.

This application is a continuation of U.S. application Ser. No. 12/605,849, filed on Oct. 26, 2009, which is a continuation of U.S. application Ser. No. 11/615,516, filed Dec. 22, 2006, the disclosures of which are both incorporated herein by reference.

### BACKGROUND

The handling, mixing and delivery of particles can be challenging. Often, one or more physical properties of the particles themselves are important to the particular application. Particulate shape, particulate size and particulate porosity. For example, often describe important physical properties or characteristics. Environmental conditions (humidity, temperature, shear forces among others) encountered by particles during use or storage can, and often do, affect one or more of the particles properties. Aggregation, agglomeration, attrition and flocculation represent some of the more common degradative effects on particles and their presence or progression greatly limits the utility of particles.

Achieving a uniform blend of particles is a problem faced daily by engineers and operators in industries as varied as pharmaceuticals, foods, plastics, ceramics processing, paints, and coatings, inks and battery production. Even when an acceptable blend is obtained, additional challenges arise in maintaining the blend through one or more pieces of downstream equipment. Poor blending or the inability to maintain an adequate blend before and during processing can lead to additional and unnecessary costs, including costs associated with rejected material and decreased yields, added blending time and energy, decreased productivities, start-up delays and defective or out-of-specification products. Powder caking of raw and in-process materials, particularly during storage (in, e.g., bags or drums) can also pose significant problems. Both powder caking and an inability to achieve uniform blends and mixtures can decrease batch uniformity which, among other drawbacks, can require increased testing and sampling.

Some flowability aids are known. For example, fumed silica is a common powder additive that can be used to improve flow characteristics. While relatively inexpensive, fumed silica often is ineffective in preventing agglomeration of many particle types. Flowability is also a matter of degree; many, if not most, uses of fumed silica lead to some agglomeration and aggregation. Some undemanding industrial applications can tolerate a level of agglomeration not tolerated in more demanding applications. Applications involving precise metering or mixing of a powder, however, require more. Even in relatively undemanding applications, the ability to improve powder flow can provide an increase in homogeneity with milder mixing conditions or with reduced mixing periods. Additionally, increased powder flowabilities can allow utilization of lower levels of expensive ingredients (e.g., dyes and pigments), particularly where the requirement of using a level of such ingredients correlates with the dispersibility of the materials in the powder with which they are mixed.

Particles handling and processing technologies today lie significantly behind the development pace of companion technologies used in liquid processes, and there remain a great many practical problems handling powders that current methods cannot effectively address. Particles exhibiting enhanced flowability and processability are desired for a wide range of applications including demanding industrial uses.

### SUMMARY

In one aspect, the present invention provides a composition comprising a plurality of particles (e.g., ceramic (i.e., glass, crystalline ceramic, glass-ceramic, and combinations thereof) and polymeric particles) and non-surface-modified nanoparticles (i.e., nanoparticles that do not have a substance reacted to the respective surfaces thereof by at least one of covalent or acid/base bonding), wherein the non-surface-modified nanoparticles are present in the composition in an amount that is at least sufficient to improve at least one of the floodability or flowability of the composition relative to the composition free of nanoparticles (wherein the flowability is the sum of the indices determined by Tests A, F, G (or H, as applicable), and I, and floodability is the sum of the indices determined by the flowability and Tests B, C, and J, as described under the Heading “Test Method for Bulk Solids Characterization by Carr Indices; ASTM D6393-99” and the Carr Indices Charts of Flowability and Floodability (below in the Examples Section)). In some embodiments of the invention, flowability is improved by at least 1, 2, 3, 4, 5, 6, 7, 8, 9, or even at least 10 percent. In some embodiments of the invention, floodability is improved by at least 2, 3, 4, 5, 6, 7, 8, 9, or even at least 10 percent.

In another aspect, the present invention provides a composition comprising a plurality of particles and non-surface-modified nanoparticles, wherein the non-surface-modified nanoparticles are present in the composition in an amount that is at least sufficient to impart to the composition substantial free flowability of the particles (wherein flowability is the sum of the indices determined by Tests A, F, G (or H, as applicable), and I under the Heading “Test Method for Bulk Solids Characterization by Carr Indices; ASTM D6393-99” and the Carr Indices Chart of Flowability (below in the Examples Section)).

Compositions according to the present invention are useful in a variety of applications where improved floodability and/or flowability of particles desirable. For example, in manufacturing uses where it is desirable to at least reduce agglomeration of solid particulate, and reduce the clogging of process equipment. Another example is at least reducing the aggregation and/or uneven distribution of pigments, for example, in polymers.

Hence, the use of the present invention may be in any of a variety of manufacturing processing and/or packaging for areas such as pharmaceuticals, foods, plastics, ceramics, paints, coatings, inks

### DETAILED DESCRIPTION

Any of a variety of nanoparticles and particles can be used to practice the present invention.

Exemplary particles include organic and/or inorganic particles. In some embodiments, the particles may comprise both organic and inorganic material (e.g., particles having inorganic cores with an outer layer of organic material thereon).

Exemplary organics include polymers, lactose, medicaments, pigments, additives, fillers, excipients (e.g., microcrystalline cellulose (and other natural or synthetic polymers)), lactose monohydrate and other sugars, exfolients, cosmetic ingredients, aerogels, foodstuffs, and toner materials. Exemplary inorganics include abrasives, metals, ceramics (including beads, bubbles, and microspheres), pigments, additives, fillers (e.g., carbon black, titanium dioxide, calcium carbonate, dicalcium phosphate, nepheline (available, for example, under the trade designation “MINEX” from Unimin Corp, New Canaan, Conn.), feldspar and wollastonite), excipients, exfolients, cosmetic ingredients and silicates (e.g., talc, clay, and sericite).

Exemplary polymers include poly(vinyl chloride), polyester, poly (ethylene terephthalate), polypropylene, polyethylene, poly vinyl alcohol, epoxies, polyurethanes, polyacrylates, polymethacrylates, and polystyrene. Polymeric particles can be made using techniques known in the art and/or are commercially available, for example, under the trade designation “POLY(VINYL CHLORIDE), SECONDARY STANDARD” from Sigma-Aldrich Chemical Company, Milwaukee, Wis.

Exemplary classes of organic pigments include phthalocyanine, diarylamide, pyrazolone, isoindolinone, isoinoline, carbazole, anthraquinone, perylene and anthrapyrimidine. Exemplary organic pigments can be made using techniques known in the art and/or are commercially available, for example, under the trade designation “ORCOBRIGHT FLUORESCENT YELLOW GN 9026” from Organic Dyestuffs Corporation, Concord, N.C. Inorganic pigments include titania, carbon black, Prussian Blue, iron oxide, zinc oxide, zinc ferrite and chromium oxide. Exemplary inorganic pigments can be made using techniques known in the art and/or are commercially available, for example, under the trade designation “BAYFERROX” from Lanxess Corporation, Akron, Ohio.

Exemplary ceramics include aluminates, titanates, zirconates, silicates, doped (e.g., lanthanides, and actinide) versions thereof, and combinations thereof. Exemplary ceramic particles can be made using techniques known in the art and/or are commercially available. Exemplary ceramic bubbles and ceramic microspheres are described, for example, in U.S. Pat. No. 4,767,726 (Marshall), and U.S. Pat. No. 5,883,029 (Castle). Examples of commercially available glass bubbles include those marketed by 3M Company, St. Paul, Minn. under the designation “3M SCOTCHLITE GLASS BUBBLES” (e.g., grades K1, K15, S15, S22, K20, K25, S32, K37, S38, K46, S60/10000, S60HS, A16/500, A20/1000, A20/1000, A20/1000, A20/1000, H50/10000 EPX, and H50/10000 (acid washed)); glass bubbles marketed, for example, by Potter Industries, Valley Forge, Pa., under the trade designation “SPHERICEL” (e.g., grades 110P8 and 60P18), “LUXSIL”, and “Q-CEL” (e.g., grades 30, 6014, 6019, 6028, 6036, 6042, 6048, 5019, 5023, and 5028); hollow glass microspheres marketed. for example, under the trade designation “DICAPERL” by Grefco Minerals, Bala Cynwyd, Pa., (e.g., grades HP-820, HP-720, HP-520, HP-220, HP-120, HP-900, HP-920, CS-10-400, CS-10-200, CS-10-125, CSM-10-300, and CSM-10-150); and hollow glass particles marketed, for example, by Silbrico Corp., Hodgkins, Ill., under the trade designation “SIL-CELL” (e.g., grades SIL 35/34, SIL-32, SIL-42, and SIL-43). Examples of commercially available ceramic microspheres include ceramic hollow microspheres marketed, for example, by Sphere One, Inc., Chattanooga, Tenn., under the trade designation, “EXTENDOSPHERES” (e.g., grades SG, CG, TG, SF-10, SF-12, SF-14, SLG, SL-90, SL-150, and XOL-200); and ceramic microspheres marketed, for example, by 3M Company under the trade designation “3M CERAMIC MICROSPHERES” (e.g., grades G-200, G-400, G-600, G-800, G-850, W-210, W-410, and W-610).

Generally, the particles will have median particle size diameters less than 200 micrometers, but greater than 100 nanometers. In some instances, the particles may have median particle size diameters less than 100 nanometers in size, but larger than the nanoparticles. In one embodiment, the particles will have median particle size diameters ranging from 0.5 micrometer to 200 micrometers, preferably from 1 micrometer to 200 micrometers, and more preferably from 1 micrometer to 100 micrometers.

Exemplary non-surface modified nanoparticles (e.g., nanospheres) include inorganic (e.g., calcium phosphate, hydroxy-apatite, metal oxides (e.g., zirconia, titania, silica, ceria, alumina, iron oxide, vanadia, zinc oxide, antimony oxide, tin oxide, and alumina-silica), metals (e.g., gold, silver, or other precious metals) and organic (e.g., insoluble sugars (e.g., lactose, trehalose (disaccharide of glucose), glucose, and sucrose), insoluble aminoacids, and polystyrene)) nanoparticles. Exemplary non-surface modified organic nanoparticles also include buckminsterfullerenes (fullerenes), dendrimers, branched and hyperbranched “star” polymers such as 4, 6, or 8 armed polyethylene oxide (available, for example, from Aldrich Chemical Company or Shearwater Corporation, Huntsville, Ala.) whose surface has been chemically modified. Specific examples of fullerenes include C₆₀, C₇₀, C₈₂, and C₈₄. Specific examples of dendrimers include polyamidoamine (PAMAM) dendrimers of Generations 2 through 10 (G2-G10), available also, for example, from Aldrich Chemical Company.

In some embodiments, the particles are the same (e.g., in terms of size, shape, composition, microstructure, surface characteristics, etc.), while in other embodiments they are different. In some embodiments, the particles have a modal (e.g., bi-modal or tri-modal) distribution. In some applications it may be desirable for the non-surface modified nanoparticles to be substantially spherical in shape. In other application, however, more elongated shapes by be desired. Aspect ratios less than or equal to 10 are considered preferred, with aspect ratios less than or equal to 3 generally more preferred.

In an exemplary embodiment, the non-surface modified nanoparticles are individual, unassociated (i.e., non-aggregated) particles that are mixed with, blended with or are otherwise distributed within the plurality of particles. While not subject to any specific physical characterization and not intending to be limited to any single characterization, one non-limiting way to identify the particles is when it is composed principally of relatively small individual particles or relatively small groups of individual particles. Generally, such particles will have a median size (generally measured as an effective diameter) of less than or equal to 1,000 micrometers, more typically less than or equal to 100 micrometers.

The particles may be distinguished from the nanoparticles by relative size, wherein the particles comprise particles that are larger than the nanoparticles. The term “nanoparticle” as used herein (unless an individual context specifically implies otherwise) will generally refer to particles, groups of particles, particulate molecules such as small individual groups or loosely associated groups of molecules, and groups of particulate molecules that while potentially varied in specific geometric shape have an effective, or average, diameter that can be measured on a nanoscale (less than 100 nanometers).

Non-surface-modified nanoparticles are commercially available, and/or can be made using techniques known in the art. Exemplary non-surface modified nanoparticles (e.g., nanospheres) include inorganic (e.g., calcium phosphate, hydroxy-apatite, metal oxides (e.g., as zirconia, titania, silica, ceria, alumina, iron oxide, vanadia, zinc oxide, antimony oxide, tin oxide, and alumina-silica), metals (e.g., gold, silver, or other precious metals) and organic (e.g., insoluble sugars (e.g., lactose, trehalose, glucose, and sucrose), insoluble aminoacids, and polystyrene) nanoparticles. Commercial sources of non-surface-modified nanoparticles include those marketed, for example, by Nalco Co, Napervillle, Ill. under the trade designation “NALCO 2326”, which is a 5 nm colloidal silica nanoparticle product. Typically, the non-surface-modified nanoparticles have an average particle size diameter less than 100 nanometers, 50 nanometers, 20 nanometers, or even less than 10 nanometers.

Additionally, non-surface modified nanoparticles may be in the form of a colloidal dispersion. Some such dispersions are commercially available, for example, as nano-sized colloidal silicas available, for example, under the product designations “NALCO 1040,” “NALCO 1050,” “NALCO 1060,” “NALCO 2327,” and “NALCO 2329” colloidal silica from Nalco Co. Metal oxide colloidal dispersions include colloidal zirconium oxide, suitable examples of which are described, for example, in U.S. Pat. No. 5,037,579 (Matchett), and colloidal titanium oxide, examples of which are described, for example, in U.S. Pat. No. 6,329,058 (Arney et al.) and U.S. Pat. No. 6,432,526 (Arney et al.).

In some embodiments of the present invention, the non-surface-modified nanoparticles are substantially associated with the surfaces of the plurality of particles.

In many cases it may be desirable for the non-surface modified nanoparticles utilized in the invention to be substantially spherical in shape. In other application, however, more elongated shapes by be desired. Aspect ratios less than or equal to 10 are considered preferred, with aspect ratios less than or equal to 3 generally more preferred. The core material will substantially determine the final morphology of the particle and thus a significant influence in selection of the core material may be the ability to obtain a desired size and shape in the final particle.

The concentration of non-surface modified nanoparticles in compositions according to the present invention will depend, for example, on the desired floodability and/or flowability of the particles therein, the effectivenesss of the non-surface modified nanoparticles (including the particular non-surface modified nanoparticles used) in providing the desired floodability and/or flowability of the particles therein, and the presence or absence of surface modified nanoparticles, or other adjuvants or excipients.

For example, the nature of the nanoparticle surface, the morphology of the particle and particle size may each influence the desired properties of the composition, the selection of the non-surface modified nanoparticles, and the amount or concentration of non-surface modified nanoparticle used. The presence of as little as 0.001 percent of non-surface modified nanoparticle by weight of a composition can achieve an improvement in flowability.

Generally, the non-surface modified nanoparticle will be present in an amount of less than or equal to 10 weight percent; in some embodiments less than or equal to 5 weight percent; less than or equal to 1 weight percent; or less than 0.1 weight percent. In some embodiments, the amount of surface-modified nanoparticles is from 0.001 to 20 percent; from 0.001 to 10 percent; from 0.001 to 1 percent; from 0.001 to 0.01 percent; or from 0.01 to 1 percent, by weight of the composition.

In many applications it may be desirable to select non-surface modified nanoparticles that are substantially spherical. It will be understood that such selection and optimization of component compositions will be within the skill of those in the art who are familiar with the physical properties required for the composition in a given use or application.

In some exemplary embodiment, the non-surface modified nanoparticles will not irreversibly associate with one another. The term “associate with” or “associating with” includes, for example, covalent bonding, hydrogen bonding, electrostatic attraction, London forces, and hydrophobic interactions.

Compositions according to the present invention will generally be prepared by mixing the particles with the non-surface modified nanoparticles using any suitable, conventional mixing or blending process. In one embodiment, the non-surface modified nanoparticles are prepared as a dispersion in an organic solvent and the mixing the particles is added to the dispersion. Typical solvents that may be employed include, for example, toluene, isopropanol, heptane, hexane, octane, and water.

In another embodiment of the disclosure, the non-surface modified nanoparticles and mixing the particles are blended as powders (e.g., dry blended).

Compositions made according to the methods described in the present invention can be used as additives to improve the flowabilty and floodabilty of powders or pellets, such as polymers, when these powders of pellets are required to be processed through an extruder. Additionally, the compositions according to the methods of the present invention can also be used to formulate medicaments when there is a need for improved dispersibility or flowability, for example, in a metered dose inhaler.

The following examples are offered to aid in the understanding of the present invention and are not to be construed as limiting the scope thereof. Unless otherwise indicated, all parts and percentages are by weight.

### EXAMPLES

Unless otherwise noted, all reagents and solvents were or can be obtained from Aldrich Chemical Co., Milwaukee, Wis.

### Test Method for Bulk Solids Characterization by Carr Indices; ASTM D6393-9

This test method is often referred to as Carr Indices. It provides measurements that can be used to describe the bulk properties of a powder or granular material.

The test method is suitable for free flowing and moderately cohesive powders and granular materials up to 2.0 mm in size. Materials must be able to pour through a 7.0±1.0-mm diameter funnel outlet when in an aerated state.

Eight measurements and two calculations provide ten tests for Carr Indices. Each individual test or a combination of several tests can be used to characterize the properties of bulk solids. These ten tests are as follows:

Test A—Measurement of Carr Angle of Repose

Test B—Measurement of Carr Angle of Fall

Test C—Calculation of Carr Angle of Difference

Test D—Measurement of Carr Loose Bulk Density

Test E—Measurement of Carr Packed Bulk Density

Test F—Calculation of Carr Compressibility

Test G—Measurement of Carr Cohesion

Test H—Measurement of Carr Uniformity

Test I—Measurement of Carr Angle of Spatula

Test J—Measurement of Carr Dispersibility

### Terminology

(i) Carr angle of difference is the difference between the Carr angle of repose and Carr angle of fall.

(ii) Carr angle of fall is an angle of repose measured from a powder heap to which a defined vibration has been given.

(iii) Carr angle of repose is a measurement from the powder heap built up by dropping the material through a vibrating sieve and funnel above a horizontal plate.

(iv) Carr angle of spatula is a measurement by which a spatula is inserted into a powder heap parallel to the bottom and then lifting it up and out of the material.

(v) Carr cohesion is a descriptive measure of interparticle forces based on the behavior of the material during sieving.

(vi) Carr compressibility is a calculation made by using Carr loose bulk density and Carr packed bulk density.

(vii) Carr dispersibility is a measurement by which a powder sample is dropped through a hollow cylinder above a watch glass and then the amount of powder collected by the watch glass is measured.

(viii) Carr dynamic bulk density is a calculated bulk density of a material. It is used to compute vibration time for the Carr cohesion measurement.

(ix) Carr loose bulk density is a measurement obtained by sieving the sample through a vibrating chute to fill a measuring cup.

(x) Carr packed bulk density is a measurement obtained by dropping a measuring cup, which is filled with the sample, a specific number of times from the same height. This is sometimes referred to as a tapped density.

(xi) Carr uniformity is a measurement calculated from the particle size distribution of the powder as measured by sieving.

### Apparatus

The Carr Index measurement instrument (obtained from Hosokawa International Inc., New York, N.Y.) included a timer, a vibrating mechanism, an amplitude gage, a rheostat, and a tapping device. The timer was used to control the duration of vibration and the number of taps. The vibrating mechanism, delivered vibration at 50 to 60 Hz to the vibration plate at an amplitude of 0.0 to 3.0 mm. The amplitude gauge was mounted on the vibration plate to measure the amplitude of the vibration (in the range of from 0.0 to 4.0 mm). The rheostat dial was used to adjust the vibration amplitude of vibration plate (in the range from 0.0 to 3.0 mm). The tapping device, consisted of tap holder and tapping lift bar (tapping pin), which lifted and free-fall dropped a measuring cup a stroke of 18.0±0.1 mm and at a rate of 1.0±0.2 taps/s. The spatula assembly consisted of a (i) spatula blade, (ii) a pan base/elevator stand, and (iii) a shocker. The spatula blade was a chrome-plated brass plate mounted on the blade receiver to retain powder while the elevator stand lowered the powder-filled pan. The dimensions of the spatula blade were 80 to 130 mm length, 22.0±0.3-mm width and 3.0±0.3-mm thick. The shocker was a sliding bushing with a mass of 110.0±1.0 g at a drop height of 150.0±10.0 mm, measured from the lower edge of the bushing to the shocker base for the measurement of angle of spatula. The total mass of the shocker assembly including the sliding bushing, pole, spatula blade, and blade receiver was 0.65±0.35 kg.

The dispersibility measuring unit consisted of container comprising (i) a shutter cover, (ii) a cylindrical glass tube, and (iii) a watch glass. The container was a hopper unit with a shutter cover at the bottom to support a powder sample. The shutter cover opened horizontally to release the powder sample which fell through the glass tube onto the watch glass. The cylindrical glass tube was located vertically 170.0±10.0 mm under the shutter cover to confine the scattering/dispersed powder. The dimension of the tube was 100.0±5.0-mm diameter and 330.0±10.0-mm length. The watch glass was centered 101.0±1.0 mm under the cylindrical glass tube to collect undispersed powder. The dimension of watch glass was 100.0±5.0-mm diameter and 2.0±0.1-mm thickness with the radius of curvature of 96.3 mm, concaved upwards.

### Accessories:

The spatula pan was a stainless steel pan with at least a 100.0-mm width, a 125.0-mm length, a 25.0 mm height, and a 1.0-mm thickness, and was used to retain powder for the preparation of the measurement of Carr angle of spatula. The scoop was a stainless steel container used to transport powder. The scraper was a stainless steel plate and was used to scrape off excess powder in the cup. The cup was a 100-ml stainless steel cylindrical container with the inside dimensions of 50.5±0.1-mm diameter and 49.9±0.1-mm height and was used for Carr bulk density measurement. The wall thickness of the cup was 1.75±0.25 mm. The interior cup walls were sufficiently smooth such that machining marks were not evident. The cup extension had an acetal polyoxy methylene (obtained from DuPont, Wilmington Del., under the trade designation “DELRIN”) extension sleeve for the 100 ml measuring cup, 55.0±0.1 mm in diameter by 48.0±1.0 mm in height. The funnel for angle of repose was a glass funnel with 55° angle bowls as measured from the horizontal, 7.0±1.0-mm bottom outlet diameter and outlet stem length 33.5 mm for the measurement of Carr angle of repose.

The stationary chute was a stainless steel conical chute with the dimensions of 75.0-mm top diameter, 55.0-mm height, and 50.0-mm bottom diameter to guide the powder flow into the measuring cup. The vibration chute was a stainless steel conical chute with the dimensions of 75.0-mm top diameter, 55.0-mm height, and 50.0-mm bottom diameter installed on the vibration plate to guide the powder flow to the stationary chute or cup extension. The sieves were certified 76.0-mm diameter stainless steel sieves with the opening of 710 micrometers, 355 micrometers, 250 micrometers, 150 micrometers, 75 micrometers, and 45 micrometers. The sieve extension was a stainless steel extension piece used as a spacer in the vibration unit when only one sieve was used. The spacer ring is a white acetal polyoxy methylene (obtained from DuPont, Wilmington Del., under the trade designation “DELRIN”) spacer inserted between sieve and vibration chute or glass funnel to protect them from damage. The sieve holding bar was a chrome-plated brass holding bar used to hold the sieve assembly on the vibration plate. The pan, with a base for tapping device, measuring cup, and shocker was a stainless steel pan (210.0-mm length, 150.0-mm width, 35.0-mm height, and 1.0-mm thickness), and was designed to accept tapping device, measuring cup and platform, as well as provide a stand base for shocker.

The platform was a chrome-plated brass circular platform with a diameter of 80.0±0.3 mm and a height of 59.0±2.0 mm, and was used for the measurement of Carr angle of repose. The shocker was a sliding bushing with a mass of 110.0±1.0 g at a drop height of 150.0±10.0 mm, measured from the lower edge of the bushing to the shocker base for the measurement of Carr angle of fall. The total mass of the shocker, platform, and pan for the measurement of angle of fall was 1.35±0.25 kg. The pan had molded-in feet so it was slightly raised from the table top. The cover, for measuring dispersibility was a removable enclosure to confine the dust of sample powder when it fell onto the watch glass for the measurement of Carr dispersibility. The balance was capable of measuring sample mass to an accuracy of ±0.01 g with a maximum of 2.0 kg. A computer was used to guide the measuring operation, collect data, calculate data, and print test results.

### Procedure

The treated nanoparticle sample was riffled carefully into portions for each of the following measurements. All the measurements were performed on a strong, horizontally-leveled laboratory bench.

### Test A Carr Angle of Repose

The following parts were placed onto the vibration plate in the following order, starting at the bottom: glass funnel, spacer ring, sieve (with 710 micrometers opening), sieve extension; and sieve holding bar. The vibration assembly was fastened with knob nuts located on both sides of sieve holding bar and the platform was centered under the glass funnel. The glass funnel was positioned 76.0±1.0 from the stem end of the glass funnel mm above the platform and 180 s on 60 Hz vibrating frequency was selected on the timer.

Approximately 250 ml of treated sample was poured over the sieve using the scoop and the vibration adjustment dial (Rheostat) was set to 0. The vibrating mechanism and timer was switched on and vibration amplitude was gradually increased (to no more than 0.2 mm at a time), by incrementally turning the vibration adjustment dial until powder starts to flow out of the end of the glass funnel and builds up on the circular platform in a conical shape. The vibration mechanism was turned off when the powder starts to fall from the edge of the platform and the powder pile was completely formed. If a conical shape was not completely formed, the powder pile was removed and previous steps were repeated. After the cone has been built up, calculate an average angle of the cone (from horizontal) in relation to the edge of the platform by the equation below. This average angle is called the Carr angle of repose.

Carr Angle of Repose=tan⁻¹ [H/R]

where:

H=Height of the powder pile, mm, and R=Radius of the circular platform, mm.

The shape of the cone was always straight.

### Test B—Carr Angle of Fall

After determining the Carr Angle of Repose as above, the shocker was placed on the shocker base and the sliding bushing was carefully raised (so that the cone will not be disturbed) to the upper end of the pole (at a drop height of 150.0±10.0 mm) and allowed to fall to give a shock to the pan. This was repeated three times. The powder layer collapsed and exhibit a smaller angle of repose. Thirty seconds after the final shock, measure the angle as described above. This new, lower angle is called Carr angle of fall.

### Test C—Calculation of Carr Angle of Difference

The Carr angle of fall was subtracted from the Carr angle of repose to obtain the Carr angle of difference.

### Test D—Carr Loose Bulk Density

The parts were placed onto the vibration plate in the following order, starting at the bottom: (i) the vibration chute, (ii) the spacer ring, (iii) the sieve with opening of 710 micrometers, (iv) sieve extension; and, (v) sieve holding bar. The vibration assembly was fastened with knob nuts located on both sides of sieve holding bar. The stationary chute was supported below the vibration chute and the pan was placed directly under the stationary chute and positioned with the measuring cup in its base. The center of the measuring cup was in alignment below the center of the stationary chute with a distance between them of 30.0±5.0 mm. The scoop was used to pour 200 to 300 ml of the powder onto the sieve and the vibration time of 30 seconds was set on timer, with the vibration adjustment dial (rheostat) set to 0. The vibrating mechanism and timer were then turned on and the amplitude of vibration was adjusted to control the powder flow rate so that the powder will fill the cup within 20 to 30 s. The vibration was terminated when the cup was filled and overflowing.

Using the scraper, excess material was scraped from the top of the cup. Weigh the cup and powder were weighed. Subtracting the empty cup mass from that of cup with powder yields a difference, when divided by 100 results in the Carr loose bulk density in g/cm³. (The cup is exactly 100 ml in volume). The previous steps were repeated three times to obtain an average value.

### Test E—Carr Packed Bulk Density

This test is also known in the field as a tapped bulk density even though the sample was dropped instead tapped.

The parts were prepared in the same order as with the measurement for Carr loose bulk density without using the stationary chute. The cup extension was placed on the top of the measuring cup. And the cup was filled to the top with the scoop with treated sample and placed on the tapping device. The timer was set for a desired tapping duration of 180 s on 60 Hz power supply). The number of taps for consistent results was determined by repetitive tests in which the relationship between the tapped bulk density and number of taps is examined. The number of taps were sufficiently large so that additional taps do not result in an increase in tapped bulk density.

The tapping device was turned on. During the tapping period, it was necessary to observe the level of the powder and, add powder to the cup extension so that the final powder level was not below the rim of the measuring cup. When the tapping was completed, the cup and its extension was removed from the tapping device scraping off excessive powder from the cup surface as described above. The cup with the packed powder was weighed and the weight of the empty cup mass was subtracted from it. That difference, divided by 100, is the Carr packed bulk density of the powder in g/ml. (The cup is exactly 100 ml in volume).

### Test F—Carr Compressibility

The Carr compressibility value (C) was calculated using the following equation from the Carr loose bulk density (L), in 5.8 and the Carr packed bulk density (P) previously determined.

C=100(P−L)/P

### Test G—Carr Cohesion

FIG. 6 of the ASTM Method designates whether to use this Test G or rather to use Test H, below.

If Test G is used, the proper sieve sizes were selected for the ASTM method. The parts were placed on the vibration plate in the following order, starting at the bottom: (i) vibration chute, (ii) spacer ring, (iii) sieve 1 (smallest opening), (iv) sieve 2 (midsize opening), (v) sieve 3 (largest opening), and (vi) the sieve holding bar. The vibration assembly was fastened with knob nuts located on both sides of sieve holding bar. The vibrating mechanism was turned on and amplitude adjusted to achieve a vibration to 1.0 mm with vibration adjustment dial. When the vibration amplitude becomes stabilized, the vibration was turned of, keeping the position of vibration adjustment dial as it was.

The timer was set according to the vibration time calculated as follows:

T(s)=20+[(1.62−W)/0.016]

W=[P−L)C/100]+L

where:

T=Vibration time (seconds)

W=Carr dynamic bulk density, g/ml,

C=Carr compressibility, %,

L=Carr loose bulk density, g/ml, and

P 5 Carr packed bulk density, g/ml.

If Carr dynamic bulk density, W, is greater than 1.6 g/ml, vibration time, T, was set at 20 s.

2.0±0.01 grams of treated sample was placed on the top sieve and the vibration mechanism was turned on. Vibration was stopped after time T, and the knob nuts were loosened and the three sieves were removed and the amount of powder retained on each sieve was weighed.

The Carr Cohesion is calculated as follows:

[(Powder mass retained on the largest sieve)/2 g]×100

[Powder mass retained on the midsize sieve)/2 g]×100×(3/5)

[Powder mass retained on the smallest sieve)/2 g]×100×(1/5)

The sum of these three calculated values gives Carr cohesion [%].

### Test H—Carr Uniformity

From the particle size distribution curve, a particle size of which 60% of the powder by volume passes the sieve (d60) and a particle size of which 10% passes the sieve (d10) was determined.

Carr uniformity is calculated below:

Carr uniformity=d60/d10

### Test I—Carr Angle of Spatula

The Carr spatula assembly was used as described above. The spatula pan was put on the pan base and the pan was raised until the pan bottom contacts the spatula. The treated sample was poured into the pan so that the spatula is completely covered with several centimeters of material (about 250 ml on the spatula). The amount of material used for each measurement was consistent, that is, same depth of material over the spatula. The pan was slowly lowered away from the spatula. This exposed the spatula with a considerable amount of material on it.

An average angle Q, of the powder pile (from horizontal) in relation to the edge of spatula by the equation below and indicate the shape of the powder pile as previously described above is calculated using the following formula:

Θ=tan⁻¹ [H/X]

where:

H=height of the powder pile on the spatula (mm) and

X=half width of the spatula (mm).

The sliding bushing was raised to the highest point of the pole (at a drop height of 150.0±10.0 mm), then dropped to give only one shock to the spatula. 30 seconds after the shock an average angle of the powder on the spatula was calculated again as described above. The mean angle of spatula before and after the shock was averaged to give the Carr angle of spatula.

### Test J—Carr Dispersibility

The apparatus was enclosed in a box to prevent ambient air currents from disturbing the measurement and to contain the powder. The Carr dispersibility measuring unit was set in place as described above. The watch glass was weighed and positioned concave upwards and centered under the glass tube. 10.0±0.01 grams of powder was weighed and placed into the hopper of the container. The shutter cover was released horizontally in 1 second, allowing the powder to fall through the glass tube and onto the watch glass. The watch glass and treated material was weighed.

Carr dispersibility value was obtained by the following calculation:

Carr dispersibility=(10 g−Mass of powder on watch glass)/10 g×100.

### Carr Indices

Table 1 lists the Carr Indices for the results of Tests A, F, G, H, and I. Summation of the Carr Indices of Tests A, F, G, (or H) and I will result in the Flowability Index.

TABLE 1

| | | | | Angle of | | | | | |
|---|---|---|---|---|---|---|---|---|---|
| Angle of | | Compressi- | | Spatula | | | | | |
| Repose | | bility | | De- | | Uniformity | | Cohesion | |
| Degree | Index | % | Index | gree | Index | No. | Index | % | Index |
| [>] <25 | 25 | <5 | 25 | <25 | 25 | 1 | 25 | | |
| 26-29 | 24 | 6-9 | 23 | 26-30 | 24 | 2-4 | 23 | | |
| 30 | 22.5 | 10 | 22.5 | 31 | 22.5 | 5 | 22.5 | | |
| 31 | 22 | 11 | 22 | 32 | 22 | 6 | 22 | | |
| 32-34 | 21 | 12-14 | 21 | 33-37 | 21 | 7 | 21 | | |
| 35 | 20 | 15 | 20 | 28 | 20 | 8 | 20 | | |
| 36 | 19.5 | 16 | 19.5 | 39 | 19.5 | 9 | 19 | | |
| 37-39 | 18 | 17-19 | 18 | 40-44 | 18 | 10-11 | 18 | | |
| 40 | 17.5 | 20 | 17.5 | 45 | 17.5 | 12 | 17.5 | | |
| 41 | 17 | 21 | 17 | 46 | 17 | 13 | 17 | | |
| 42-44 | 16 | 22-24 | 16 | 47-59 | 16 | 14-16 | 16 | | |
| 45 | 15 | 25 | 15 | 60 | 15 | 17 | 15 | <6 | 15 |
| 46 | 14.5 | 26 | 14.5 | 61 | 14.5 | 18 | 14.5 | 6-9 | 14.5 |
| 47-54 | 12 | 27-30 | 12 | 62-74 | 12 | 19-21 | 12 | 10-29 | 12 |
| 55 | 10 | 31 | 10 | 75 | 10 | 22 | 10 | 30 | 10 |
| 56 | 9.5 | 32 | 9.5 | 76 | 9.5 | 23 | 9.5 | 31 | 9.5 |
| 57-64 | 7 | 33-36 | 6 | 77-89 | 7 | 24-26 | 7 | 32-54 | 7 |
| 65 | 5 | 37 | 5 | 90 | 5 | 27 | 5 | 55 | 5 |
| 66 | 4.5 | 38 | 4.5 | 91 | 4.5 | 28 | 4.5 | 56 | 4.5 |
| 67-89 | 2 | 39-45 | 2 | 92-99 | 2 | 29-35 | 2 | 57-59 | 2 |
| 90 | 0 | >45 | 0 | >99 | 0 | >35 | 0 | >79 | 0 |

Table 2 lists the Carr Indices for the flowability index (obtained from summing the values from Table 1), and Tests B, C, and J. Summation of the Carr index assigned to the Flowability Index and the Carr indices of Tests B, C, and J will result in the Floodability Index. Adding the Flowability Index and the Floodability Index will provide the total Carr Index for the solid.

TABLE 2

| | | | | Angle of | | | |
|---|---|---|---|---|---|---|---|
| Flowability | | Angle of Fall | | Difference | | Dispersibility | |
| Degree | Index | % | Index | Degree | Index | % | Index |
| >60 | 25 | <10 | 25 | >30 | 25 | <50 | 25 |
| 59-56 | 24 | 11-19 | 23 | 29-28 | 24 | 49-44 | 24 |
| 55 | 22.5 | 20 | 22.5 | 27 | 22.5 | 43 | 22.5 |
| 54 | 22 | 21 | 22 | 26 | 22 | 42 | 22 |
| 53-50 | 21 | 22-24 | 21 | 25 | 21 | 41-36 | 21 |
| 49 | 20 | 25 | 20 | 24 | 20 | 35 | 20 |
| 48 | 19.5 | 26 | 19.5 | 23 | 19.5 | 34 | 19.5 |
| 47-45 | 19.5 | 27-29 | 18 | 22-20 | 18 | 33-29 | 18 |
| 44 | 19.5 | 30 | 17.5 | 19 | 17.5 | 28 | 17.5 |
| 43 | 19.5 | 31 | 17 | 18 | 17 | 27 | 17 |
| 42-40 | 19.5 | 32-39 | 16 | 17-16 | 16 | 26-21 | 16 |
| 39 | 19.5 | 40 | 15 | 15 | 15 | 20 | 15 |
| 38 | 14.5 | 41 | 14.5 | 14 | 14.5 | 19 | 14.5 |
| 37-34 | 12 | 42-49 | 12 | 12 | 12 | 18-11 | 12 |
| 33 | 10 | 50 | 10 | 10 | 10 | 10 | 10 |
| 32 | 9.5 | 51 | 9 | 9.5 | 9.5 | 9 | 9.5 |
| 31-29 | 8 | 52-56 | 8 | 8 | 8 | 8 | 8 |
| 28 | 6.25 | 57 | 7 | 6.25 | 6.25 | 7 | 6.25 |

### Comparative Example A

95 grams of nepheline syenite (obtained from Unimin Corporation, under the trade designation “MINEX 7”) was characterized using the “Standard Test Method for Bulk Solids Characterization by Carr Indices; ASTM D6393-99” (described above) using Test A, B, C, D, E, F, G, I, and J. The Carr Index was derived after the methods described by Carr in Chemical Engineering vol. 72, pp. 163-168 (1965), the disclosure of which is incorporated herein by reference. The results are reported in Table 3, below.

TABLE 3

| Test | Example | Comp. A | 1 | 2 | 3 |
|---|---|---|---|---|---|
| A | Angle of Repose (°, Index) | 45.9 (14.5) | 49.6 (12) | 45.6 (14.5) | 44.0 (16) |
| B | Angle of Fall (°, Index) | 34.2 (16) | 32.3 (16) | 30.3 (17.5) | 36.0 (16) |
| C | Angle of Difference (°, Index) | 11.7 (12) | 17.3 (16) | 15.3 (15) | 8.0 (8) |
| D | Loose Bulk Density (g/cm³) | 0.432 | 0.407 | 0.424 | 0.487 |
| E | Packed Bulk Density(g/cm³) | .957 | 0.884 | 0.917 | 0.935 |
| F | Compressibility (%, Index) | 54.9 (0) | 54.0 (0) | 53.8 (0) | 47.9 (0) |
| G | Cohesiveness (%, Index) | 75.7 (2) | 72.3 (2) | 75.3 (2) | 18.1 (12) |
| I | Angle of Spatula (°, Index) | 57.5 (16) | 59.5 (15) | 54.3 (16) | 54.1 (16) |
| J | Dispersibility (%, Index) | 2.60 (3) | 14.3 (12) | 27.2 (17) | 40.8 (21) |
| | Flowability Index | 32.5 | 29 | 32.5 | 44 |
| | Floodability Index | 40.5 | 52 | 59.5 | 62.5 |
| | Total Index | 73 | 81 | 92 | 106.5 |
| | (Carr Index) | | | | |

### Example 1

6.7 grams of colloidal silica nanoparticles (5 nm; 15% by weight solids, available from Nalco Co., Naperville, Ill., under the trade designation “NALCO 2326”) were added to 200 grams of nepheline syenite (“MINEX 7”), and manually kneaded in a plastic bag for 5 minutes until completely blended, and then dried in an oven at 100° C. for 3 hours.

The resulting treated nepheline syenite was characterized as described in Comparative Example A, and the results reported in Table 3, above.

### Example 2

13.3 grams of colloidal silica nanoparticles (5 nm; 15% by weight solids; “NALCO 2326”) were added to 200 grams of nepheline syenite (“MINEX 7”), and manually kneaded in a plastic bag for 5 minutes until completely blended, and then dried in an oven at 100° C. for 3 hours.

The resulting treated nepheline syenite was characterized as described in Comparative Example A, and the results reported in Table 3, above.

### Example 3

26.7 grams of colloidal silica nanoparticles (5 nm; 15% solids, “NALCO 2326”) were added to 200 grams of nepheline syenite (“MINEX 7”) and manually kneaded in a plastic bag for 5 minutes until completely blended, and then dried in an oven at 100° C. for 3 hours.

The resulting treated nepheline syenite was characterized as described in Comparative Example A, and the results reported in Table 3, above.

### Comparative Example B

95 grams of ceramic microspheres (obtained from 3M Company, St. Paul, Minn., under the trade designation “3M W410 ZEOSPHERES”) was characterized as described in Comparative Example A, and the results reported in Table 4, below.

TABLE 4

| Test | Example | Comp. B | 4 |
|---|---|---|---|
| A | Angle of Repose (°, Index) | 50.5 (12) | 49.5 (12) |
| B | Angle of Fall (°, Index) | 36.8 (16) | 29.3 (18) |
| C | Angle of Difference (°, Index) | 13.7 (14.5) | 20.2 (18) |
| D | Loose Bulk Density (g/cm³) | 0.458 | 0.677 |
| E | Packed Bulk Density(g/cm³) | 1.035 | 1.132 |
| F | Compressibility (%, Index) | 55.7 (0) | 40.2 (2) |
| G | Cohesiveness (%, Index) | 59.9 (2) | 8.9 (14.5) |
| I | Angle of Spatula (°, Index) | 55.2 (16) | 58.7 (16) |
| J | Dispersibility (%, Index) | 27.9 (17.5) | 56.7 (25) |
| | Flowability Index | 30 | 44.5 |
| | Floodability Index | 56 | 78.5 |
| | Total Index | 86 | 123 |
| | (Carr Index) | | |

Further, the packing factor was determined as follows. A fully automated gas displacement pycnometer obtained under the trade designation “ACCUPYC 1330 PYCNOMETER” from Micromeritics, Norcross, GAa, was used to determine the true density (g/cm³) of the composite material and glass residual according to ASTM D-2840-69, “Average True Particle Density of Hollow Microspheres,” the disclosure of which is incorporated herein by reference.

Using a tap-pak volumeter (obtained under the trade designation “JEL” Tap-Pak Volumeter, from J. Engelsmann A G, Ludwigschafen, Germany), a known weight of sample, wtₛₐₘₚₗₑ, to be tested was poured into a graduated cylinder and tapped for 3,000 cycles. The bulk volume, V_{bulk}, was read off of the graduated cylinder to the nearest 5 cm³. The bulk density was determined using the following equation:

Bulk Density(g/cm³)=wtₛₐₘₚₗₑ/V_{bulk}.

The Packing Factor, in turn was determined using the following equation:

Packing Factor(%)=(Bulk Density/True Density)×100.

The packing factor was 3.5%.

### Example 4

26.7 grams of colloidal silica nanoparticles (5 nm; 15% solids, “NALCO 2326”) were added to 200 grams of ceramic microspheres (obtained from 3M Company under the trade designation “3M W410 ZEOSPHERES”) and manually kneaded in a plastic bag for 5 minutes until completely blended, and then dried in an oven at 100° C. for 3 hours.

The resulting treated ceramic microspheres were characterized as described in Comparative Example B, and the results reported in Table 4, above. Further, the packing factor was 41.9%.

### Comparative Example C

95 grams of hollow glass microspheres identical to those (the same as that marketed by 3M Company under the trade designation “3M S60HS GLASS MICROSPHERES”, except for the absence of an anti-caking agent that is present on the microspheres sold commercially) was characterized as described in Comparative Example B, and the results reported in Table 5, below. Further, the packing factor was 34.1%.

TABLE 5

| Test | Example | Comp. C | 5 | 6 |
|---|---|---|---|---|
| A | Angle of Repose (°, Index) | 55.1 (10) | 44.8 (15) | 43.1 (16) |
| B | Angle of Fall (°, Index) | 51.5 (8) | 28.8 (18) | 31.2 (17) |
| C | Angle of Difference (°, Index) | 3.6 (3) | 16.0 (16) | 11.9 (12) |
| D | Loose Bulk Density (g/cm³) | 0.069 | 0.151 | 0.218 |
| E | Packed Bulk Density(g/cm³) | 0.201 | 0.297 | 0.333 |
| F | Compressibility (%, Index) | 65.7 (0) | 49.2 (0) | 34.5 (7) |
| G | Cohesiveness (%, Index) | 50.0 (7) | 0.3 (15) | 0 (15) |
| I | Angle of Spatula (°, Index) | 50.3 (16) | 62.4 (12) | 58.2 (16) |
| J | Dispersibility (%, Index) | 23.3 (16) | 75.9 (25) | 75.7 (25 |
| | Flowability Index | 33 | 42 | 54 |
| | Floodability Index | 37 | 75 | 76 |
| | Total Index | 70 | 117 | 130 |
| | (Carr Index) | | | |

### Example 5

2.7 grams of colloidal silica nanoparticles (5 nm; 15% solids, “NALCO 2326”) were added to 200 grams of hollow glass microspheres (the same as that marketed by 3M Company under the trade designation “3M S60HS GLASS MICROSPHERES” except for the absence of the anti-caking agent that is present on the microspheres sold commercially) and manually kneaded in a plastic bag for 5 minutes until completely blended. The ensuing mixture was dried in an oven at 100° C. for 3 hours.

The resulting treated hollow glass microspheres were as described in Comparative Example B, and the results reported in Table 5, above. Further, the packing factor was 38.2%.

### Example 6

26.7 grams of colloidal silica nanoparticles (5 nm; 15% solids, “NALCO 2326”) were added to 200 grams of hollow glass microspheres (the same as that marketed by 3M Company under the trade designation “3M S60HS GLASS MICROSPHERES”, except for the absence of the anti-caking agent is present on the microspheres sold commercially) and manually kneaded in a plastic bag for 5 minutes until completely blended. The ensuing mixture was dried in an oven at 100° C. for 3 hours.

The resulting treated hollow glass microspheres were characterized as described in Comparative Example B, and the results reported in Table 5, above. Further, the packing factor was 48.8%.

### Example 7

1.9 gram of colloidal silica nanoparticles (5 nm; 15% by weight solids, “NALCO 2326”) were added to 300 grams of calcium carbonate (CaCO₃, 10 micrometers; obtained from Sigma-Aldrich, Milwaukee, Wis.), and manually kneaded in a plastic bag for 1 hour until completely blended, and then dried in an oven at 120° C. for 3 hours.

The resulting treated calcium carbonate was characterized as described in Comparative Example A, and the results reported in Table 6, below.

TABLE 6

| Test | Example | 7 | 8 | 9 |
|---|---|---|---|---|
| A | Angle of Repose | 48.8 (12) | 40.5 (17.5) | 39.9 (17.5) |
| | (°, Index) | | | |
| B | Angle of Fall | 41.8 (12) | 39.1 (16) | 37.8 (16) |
| | (°, Index) | | | |
| C | Angle of Difference | 7 (6.25) | 0 (0) | 2.1 (3) |
| | (°, Index) | | | |
| D | Loose Bulk Density | 0.345 | 0.92 | 0.556 |
| | (g/cm³) | | | |
| E | Packed Bulk Density | 0.829 | 1.207 | 0.632 |
| | (g/cm³) | | | |
| F | Compressibility | 58.4 (0) | 23.8 (16) | 12 (21) |
| | (%, Index) | | | |
| G | Cohesiveness | 97.5 (0) | 3.4 (15) | 36.9 (7) |
| | (%, Index) | | | |
| I | Angle of Spatula | 59.2 (16) | 50.5 (16) | 43.9 (18) |
| | (°, Index) | | | |
| J | Dispersibility | 10.1 (10) | 59.8 (25) | 28.0 (17.5) |
| | (%, Index) | | | |
| | Flowability Index | 51 | 87.5 | 86.5 |
| | Floodability Index | 49.25 | 66.0 | 61.5 |
| | Total Index | 100.25 | 153.5 | 148 |
| | (Carr Index) | | | |

### Example 8

Example 8 was prepared as described in Example 7, except the 10 micrometer CaCO₃ was replaced with 300 grams of 5 micrometer CaCO₃ (obtained from Alfa Aesar, Ward Hill, Mass.).

The resulting treated calcium carbonate was characterized as described in Comparative Example A, and the results reported in Table 6, above.

### Example 9

Example 9 was prepared as described in Example 7, except the 10 micrometer CaCO₃ was replaced with 300 grams of poly (vinyl chloride) (obtained from Sigma-Aldrich, inherent viscosity 0.725).

The resulting treated calcium carbonate was characterized as described in Comparative Example A, and the results reported in Table 6, above.

Various modifications and alterations of this invention will be apparent to those skilled in the art without departing from the scope and spirit of this invention, and it should be understood that this invention is not limited to the illustrative embodiments set forth herein.

What is claimed is:

1. A composition comprising a plurality of particles and non-surface-modified nanoparticles, wherein the non-surface-modified nanoparticles are in the form of a colloidal dispersion and present in the composition in an amount of from 0.001 to 20 percent by weight, based on the total weight of the composition.

2. The composition according to claim 1, wherein the plurality of particles includes at least one of polymeric particles, glass particles, ceramic bubbles, or ceramic microspheres.

3. The composition according to claim 1, wherein the plurality of particles has a median particle size diameter less than 200 micrometers.

4. The composition according claim 1, wherein the non-surface-modified nanoparticles have an average particle size diameter less than 100 nanometers.

5. The composition according claim 1, wherein the non-surface-modified nanoparticles have an average particle size diameter less than 50 nanometers.

6. The composition according claim 1, wherein the non-surface-modified nanoparticles have an average particle size diameter less than 20 nanometers.

7. The composition according claim 1, wherein the non-surface-modified nanoparticles have an average particle size diameter less than 10 nanometers.

8. The composition according claim 1, wherein the amount of non-surface-modified nanoparticles is in a range from 0.001 to 10 percent by weight, based on the total weight of the composition.

9. The composition according claim 1, wherein the amount of non-surface-modified nanoparticles is in a range from 0.001 to 1 percent by weight, based on the total weight of the composition.

10. The composition according claim 1, wherein the amount of non-surface-modified nanoparticles is in a range from 0.001 to 0.01 percent by weight, based on the total weight of the composition.

11. The composition according claim 1, wherein the non-surface-modified nanoparticles comprise an inorganic material selected from the group consisting of: alumina, calcium phosphate, iron oxide, silica, zinc oxide, zirconia, and combinations thereof.

12. The composition according claim 1, wherein the non-surface-modified nanoparticles comprise an organic material.
